(19) United States
(12) Patent Application Publication
(10) Pub. No.: US 2007/0243604 A1
(43) Pub. Date: Oct. 18, 2007

(54) Marker for Selecting Transformant with The Use of Lethal Gene

(76) Inventors: Masayuki Machida, Ibaraki (JP); Haruhiko Masaki, Tokyo (JP); Sumiko Kunihiro, Ibaraki (JP); Hiroko Hagiwara, Ibaraki (JP)

Correspondence Address: MORGAN LEWIS & BOCKIUS LLP, 1111 PENNSYLVANIA AVENUE NW, WASHINGTON, DC, 20004, US

(21) Appl. No.: 10/522,366

(22) PCT Filed: Jul. 28, 2003
(86) PCT No.: PCT/JP03/09543

§ 371 Date: Sep. 26, 2005

(30) Foreign Application Priority Data
Jul. 26, 2002 (JP) P. 2002-218735

(51) Int. Cl. C12N 15/00; C07H 21/04

(52) U.S. Cl. 435/320.1; 536/23.1

(57) ABSTRACT

A DNA fragment prepared by inserting a translation termination codon into 5′ upstream side of the active site of a lethal gene, a transformant selection marker which uses the same, and a vector into which the marker is inserted. Since this lethal gene is used as a gene marker, complete extinction of transformants having no exogenous gene can be achieved, and a transformant selection marker capable of effecting stable amplification of a vector containing an exogenous gene in a host can be obtained. In addition, a vector which can effect accurate and efficient gene analyses by DNA microarray and the like is obtained.

### TECHNICAL FIELD

The present invention relates to a DNA fragment useful as a marker for transformant selection, a vector into which the DNA fragment is inserted, and a marker for transformant selection comprising the DNA fragment.

### BACKGROUND ART

Conventionally, when an appointed transformant is obtained by inserting an exogenous gene into a vector and transforming a host with it, various gene markers are used for selecting a transformant of interest alone. For example, when a β-galactosidase gene is used as the marker, the gene is conjugated with an exogenous gene and inserted into a vector, and a host is transformed with it. While a β-galactosidase gene is expressed by a transformant harboring the exogenous gene, β-galactosidase gene is not expressed by one other than the transformant. Accordingly, the desired transformant can be selected by detecting the expression of a β-galactosidase gene as a change in color of colonies based on the structural change of a coloring substance added to the medium (Sanbrook et al. (1989) Molecular Cloning—A Laboratory Manual, 2nd ed., 1.85-1.86).

Also, a method which uses a lethal gene such as a topoisomerase or colicin E1 gene as the gene marker is also known (JP-A-57-139095). In this method, an exogenous gene is inserted into the translation region of a lethal gene, so that expression of the gene is inhibited, and only a clone harboring the exogenous gene is selectively grown. However, in the case of selection by coloring using a β-galactosidase gene or the like, not only it is necessary to add a coloring substance such as X-gal to the medium, but also transformants not harboring the insertion fragment are also grown, so that a large area of the agar medium is required for isolating a large number of transformants. On the other hand, in the case of using a lethal gene, the transformants not harboring the insertion fragment die out, so that it is possible to reduce the medium area for isolating transformants or to carry out the selection by using a liquid medium. However, when lethality of the lethal gene is too high, (1) mutation is introduced into the lethal gene at a high frequency during the culturing, so that the lethality cannot be maintained stably, and (2) it is necessary to use a host into which an inactivated gene or mutation is introduced, for regulating toxicity of the lethal gene in amplifying the vector. Also, when lethality of the lethal gene is low, a promoter having high expression activity is necessary for exerting the lethality by over-expression.

In addition, when a library is constructed by using a plasmid vector, a phage vector or the like, complete digestion using excess amounts of restriction enzymes is important for the purpose of improving existing frequency of insertion fragments of clones of the library. On the other hand, the complete digestion using excess amounts of restriction enzymes induces reduction of the number of independent clones constituting the library and pseudopositive of the inserted marker of a fragment such as lacZ due to deletion of a terminal base, caused by the presence of other nuclease activities such as an exonuclease activity contaminated in the restriction enzymes. Thus, there are many cases in which excess digestion with restriction enzymes cannot be carried out for securing the maximum number of independent clones constituting the library. In such cases, secure extinction of the clones having no insertion fragment is most effective, and when this is achieved, it becomes possible to prepare a high quality library having a large number of independent clones constituting the library, without reducing insertion frequency of insertion fragments of clones.

### DISCLOSURE OF THE INVENTION

An object of the present invention is to provide a transformant selection marker by using a lethal gene as a gene marker which can attain complete extinction of transformants having no exogenous gene and also can effect stable amplification of a vector containing the exogenous gene in the host, particularly, to provide a convenient means for optionally controlling activity of the lethal gene in response to the degree of resistance of each host against the lethal gene, and thereby to solve the above-described problems involved in the prior art.

As a result of intensive studies, the present inventors have found that the above-described objects can be solved by inserting one or two or more translation termination codons into the 5′ upstream side of a lethal gene and using it as a marker for transformant selection, and thus the present invention has been accomplished.

That is, the present invention relates to the following (1) to (12).

(1) A DNA fragment in which a translation termination codon is inserted into the 5′ upstream of an active site of a lethal gene.

(2) The DNA fragment according to the above-described (1), which has restriction enzyme cleavage sites in both terminal sides.

(3) The DNA fragment according to any one of the above-described (1) to (3), wherein one or at least two translation termination codons are inserted.

(4) The DNA fragment according to any one of the above-described (1) to (3), wherein the active site encodes a colicin-derived polypeptide.

(5) The DNA fragment according to any one of the above-described (1) to (4), wherein the active site comprises a nucleotide sequence encoding the amino acid sequence represented by SEQ ID NO:18 or 19.

(6) A DNA fragment which comprises the nucleotide sequence represented by SEQ ID NO:14.

(7) The DNA fragment according to any one of the above-described (1) to (6), wherein a neutralizing gene is conjugated to the 3′ downstream side of the active site of the lethal gene.

(8) The DNA fragment according to the above-described (7), wherein the nucleotide sequence of the neutralizing gene is represented by SEQ ID NO:15.

(9) A marker for transformant selection, which comprises the DNA fragment according to any one of the above-described (1) to (8).

(10) The marker for transformant selection according to the above-described (9), wherein the transformant is obtained by transforming Escherichia coli.

(11) A recombinant vector into which the DNA fragment according to any one of the above-described (1) to (8) is inserted.

(12) The recombinant vector according to the above-described (11), which is free of an expression promoter for the lethal gene.

### BEST MODE FOR CARRYING OUT THE INVENTION

When the host is Escherichia coli, examples of the lethal gene which constitutes the DNA fragment to be used in the present invention as a marker for selecting transformant include E1, E2, E3, E4, E5, E6, E7, E8, E9, Ia, Ib, D, B, A, M, N and K of colicin, cloacin DF13, A1, A2 A3 of clebicin, AP41, S1, S2, S3 and S4 of pyocin, barnase, pemK and the like. Also, when the host is enteric bacterium other than E. coli such as Enterobacter, Pseudomonas aeruginosa, the genus Bacillus or the like, the above substances or homologues thereof can be used for the same purpose. As the neutralizing gene which corresponds to the immunity E3, inhibitors for respective lethal genes (respective immunity genes for colicin, cloacin, clebicin and pyocin; barstar gene for barnase; and pemI gene for pemK) can be used. A gene encoding a killer toxin can be used for yeast, and a small peptide of about 50 amino acids and a phage-like bacteriocin can be used for Gram-positive bacteria such as lactic acid bacteria. Although a neutralizing gene for killer toxin is not specified, its inactivated gene can be used for lactic acid bacterial bacteriocin. The range of biological species to which the present invention is applicable is not limited to the above, and it can be applied to all of the other biological species including microorganisms, fungi, plants, animals and the like to which lethal genes are applicable.

According to the present invention, only the active site of these lethal genes is used by artificially taking it out to shorten the gene size, and one or plural translation termination codons (TAG, TGA and TAA) are inserted into the 5′ upstream side this active site to obtain a DNA fragment to be used as a transformant selection marker.

The lethality activity of the above-described lethal gene is controlled by the number of translation termination codons to be inserted. In addition, although the suppressor intensity for termination codons possessed by hosts is varied, the most suitable marker for each host can be prepared by controlling the number of translation termination codons in response to this suppressor intensity. For example, when a lethal gene having extremely strong lethality is used, the number of translation termination codons to be inserted is increased, and when the suppressor intensity of the host to be transformed is also high, the number of the translation termination codons is further increased. On the contrary, even when the lethal activity of the lethal gene is high, the number of the translation termination codons to be inserted is reduced when a host having low suppressor intensity is used. That is, according to the present invention, the number of the translation termination codons to be inserted is decided in view of both sides of the lethal activity of the lethal gene to be inserted and the suppressor activity strength of the host.

In addition, according to the present invention, for example, when the active site of a lethal gene having extremely high lethal activity such as colicin is used, a DNA fragment having a neutralizing gene (immunity gene) for the lethal gene, in addition to the translation termination codon, can be prepared and used as the transformant selection marker. By such a lethal activity reducing means, it becomes possible to use an E. coli strain which is sensitive to the toxicity of lethal gene. In addition, this means to use a neutralizing gene is also effective when a vector having high lethal gene expression is used. Also, as a lethal activity reducing means, a means of not using an expression promoter for the DNA fragment to be inserted as the selection marker is also effective, and in that case, it is not necessary to take functional relationship of the vector DNA with translation reading frame or the like into consideration, so that designing of a vector having considerably high degree of freedom becomes possible.

The insertion of the translation termination codon according to the present invention provides a particularly advantageous result when a gene having high lethal activity such as colicin is used. That is, as described in the above, when such a lethal gene having high lethal activity is used, mutation is induced at a high frequency in the lethal gene during culturing to increase resistance of the host, so that a host which does not have the exogenous gene also grows, and, as a result, the selection efficiency of the transformant of interest by the selection marker is reduced. However, in the case of the present invention, it becomes possible to inhibit mutation of the lethal gene and also to control the lethal activity of the lethal gene artificially and appropriately in such a manner that transformants having no exogenous gene can be wiped out, due to the insertion of translation termination codon and adjustment of the number thereof to be inserted. Also, in addition to this, since the active site of a lethal gene originally having high lethal activity is used, it is not necessary to locate it in the downstream of a strong promoter, or carry out fusion with other peptide, for the purpose of reinforcing expression of the lethal gene, so that a transformant selection marker DNA most suitable for each host can be prepared by a convenient means.

The DNA fragment to be used in the selection marker of the present invention is described further illustratively, with reference to a case in which colicin E3 gene is used. Colicin E3 is an antibacterial polypeptide as a member of bacteriocin produced by E. coli, and its gene is present on a plasmid. Complete length gene of the plasmid (plasmid ColE3-CA38) is shown in SEQ ID NO:16 of the Sequence Listing. In the gene, a nucleotide sequence from the 331st to 1986th positions (including termination codon) is the structural gene moiety of colicin E3, the structural gene moiety of the neutralizing gene (immunity gene) E3 is present in a nucleotide sequence from the 1996th to 2253rd positions, and the structural gene moiety of the neutralizing gene E8 is present in a nucleotide sequence from the 2420th to 2677th positions.

An amino acid sequence which corresponds to this colicin E3 gene is shown in SEQ ID NO:17. The active site of colicin E3 is a moiety from the 442nd position alanine (corresponds to the 1654th to 1656th position GCT of SEQ ID NO:16) of the amino acid sequence represented by SEQ ID NO:17 or from the 455th position lysine (corresponds to the 1693rd to 1695th position AAA of the same) to the 551st position leucine (corresponds to the 1081st to 1983rd position CTT of the same), and a DNA fragment encoding this amino acid sequence moiety is used as the marker gene. Amino acid sequences of the colicin active site starting from the above-described alanine and lysine are shown in SEQ ID NOs:18 and 19, respectively. According to the present invention, those which have nucleotide sequences encoding these amino acid sequences can be used, and the nucleotide sequences in which one or two or more bases are deleted, substituted or added can also be used, so long as they show lethality activity upon the host.

A translation termination codon (TAG; amber termination codon) is arranged in the 5′ upstream of the above-described active site, and restriction enzyme cleavage sites are arranged in the upstream of this termination codon and in the downstream side of the 3′-terminal termination codon of the active site. Also, as occasion demands, a neutralizing gene (immunity gene) is added to the downstream side of the 3′-terminal side restriction enzyme cleavage site. Although nucleotide sequence of this neutralizing gene for colicin E3 is shown in SEQ ID NO:15, the nucleotide sequence in which one or two or more bases are deleted, substituted or added can also be used with the proviso that it has the neutralizing activity for the lethal gene to be used. Nucleotide sequence of the DNA fragment constructed in this manner to be used as the transformant selection marker is shown in SEQ ID NO:20, and in the sequence, a translation termination codon (TGA) is arranged at tree positions in the 5′ upstream of the above-described active site, and two SfiI restriction enzyme cleavage sites are arranged in such a manner that their protruding terminals have different sequences.

A case in which colicin E3 gene is used was described in the above, but it can be easily understood in view of its principle that the means of the present invention for adding termination codon is not limited to the above-described example but has broad universality.

When a lethality gene is introduced into E. coli or the like for the purpose of shortening the lethality gene to be used and adding translation termination codon in preparing a DNA fragment to be used as the selection marker in the present invention, it is necessary in general to carry out it in such a manner that the neutralizing gene of the lethality gene can be expressed in the E. coli. For this purpose, the neutralizing gene is allowed to coexist on a vector to be used in introducing the lethality gene so that it can be expressed, or a plasmid or the like constructed in advance for expression of the neutralizing gene is introduced into the E. coli. After constructing a lethal gene into which a desired number of termination codon is inserted, a DNA fragment containing the lethal gene is cleaved by using a restriction enzyme, and the DNA fragment is separated and recovered by using an appropriate means such as electrophoresis. This DNA fragment is finally ligated by using a ligase or the like to the corresponding restriction enzyme site of a vector to be used in the preparation of a library or the like, and transformed into an E. coli to be used in the amplification.

In this connection, it is necessary that the E. coli to be used in the amplification has a suppressor mutation weaker than the E. coli to be finally used as the host for the library construction or the like, or has a gene which neutralizes the lethal gene in advance. Also, the E. coli to be used in the amplification may be the same as the E. coli to be finally used as the host, but in that case, it is necessary that the expression strength of the lethal gene on the vector can be controlled at the transcription level or the like by an appropriate inducer (induction condition), an inhibitor (inhibition condition) or the like. In this case, when the vector is amplified, expression of the lethality gene is inhibited by the above-described method, or when it is finally used for a purpose such as final construction of a library, its expression is induced by the above-described method. It is possible to stably amplify the lethality gene into which a suitably number of terminal codons are inserted by any one of the above-described methods, and it can kill the host effectively when it is used for a final purpose such as construction of a library.

When a vector is constructed by using the DNA fragment of the present invention as the selection marker, there are methods in which (1) similar to the general galactose fragment and the like, a single restriction enzyme cleavage site is inserted between the translation initiation codon and the active site, or into the active site, of the DNA fragment to bind to the vector, and the selection marker is inactivated by inserting an exogenous gene fragment into this insertion site, or (2) the vector is cleaved at 2 positions to form two different protruding terminals, the DNA fragment of the present invention is inserted in advance into the resulting cloning site, and then an exogenous gene fragment is inserted into this part in a substituted manner. According to the present invention, any of these methods can be used, and among these two methods, the method of (1) realizes the restriction enzyme cleavage site at one position, but deletion of one or more bases occasionally occurs due to exonuclease activity and the like contaminated in the restriction enzyme, and in that case, a lethal gene as the marker gene is inactivated due to deletion of amino acid residues necessary for the frameshift of translation and activity, even when the exogenous gene fragment is not inserted into the cloning site, so that pseudopositive is formed and effective selection sometimes becomes possible. On the other hand, the method of (2) requires two restriction enzyme cleavage sites, but the problem of causing pseudopositive by the frameshift of translation does not occur, so that the method of (2) is desirable.

The vector to be used may be any one of plasmid, phage, cosmid and the like with no particular limitation.

In addition, when the vector is constructed by the above-described insertion of two restriction enzyme cleavage sites, continuation of translation from the upstream of the cleavage sites is not required, and both of the translation initiation and termination codons of the lethal gene active region can be arranged in the DNA fragment of the present invention, so that a translation initiation codon is not necessary in the upstream of the cloning site. Thus, when a translation initiation codon is not arranged in the upstream of the cloning site, it becomes possible also to control expression of the cloned insertion fragment at a markedly low level. Accordingly, easy cloning can be realized even when the exogenous gene has strong toxicity to the host cell. However, the exogenous gene can be expressed as a matter of course by arranging a translation initiation codon in the cloning site, and in that case, transformants having a vector into which the exogenous gene is not inserted die out so that the exogenous gene alone can be expressed. In addition, when the desired DNA fragment is inserted into the vector and obtained as a clone, all of the lethal gene moieties according to the present invention are removed, so that there is no interference of biological functions between the inserted gene and selection marker, and the degree of freedom in designing the vector is high. Also, since the size of the vector after the gene insertion can be shortened, efficiency of transformation and amplification in the host cell is high.

On the other hand, a demerit by the insertion of two restriction enzyme cleavage sites is that the efficiency is reduced when the amount of the insertion fragment is too large. However, when the amount of the insertion fragment is decreased in order to prevent this, the number of clones having a vector which is re-ligated due to no insertion of the exogenous gene fragment increases. In order to decrease the number of re-ligation clones, it is necessary to carry out dephosphorylation by an alkaline phosphatase treatment or recover the vector DNA fragment from a gel by electrophoresis, but even if the ratio of the re-ligation clones can be decreased by this, the number of independent clones constituting the library is sharply decreased in general. On the other hand, since the vector of the present invention is constructed in such a manner that protruding terminals of the two restriction enzyme cleavage sites of the vector are different from each other, and the lethal gene is arranged in the fragment interposed between these restriction enzyme cleavage sites, the re-ligation clones formed during the insertion of an exogenous gene fragment into the vector do not contain the exogenous gene fragment, but contain the active site of the lethal gene, so that the re-ligated clones die out by the expression of this active site of the lethal gene and can be specifically removed. In addition, because of this, it becomes possible to improve existing frequency of clones into which an exogenous gene is efficiently inserted by decreasing the amount of insertion fragment of the exogenous gene, and different from the conventional method, it is not necessary to use excess amounts of restriction enzymes in order to improve existing frequency of the clones.

When a transformant having an insertion fragment is selected, the selection is generally carried out by allowing transformants to grow on an agar medium and to form colonies. This is because it is necessary to judge the presence of the insertion fragment, for example, based on the presence or absence of coloring of colonies on the agar medium containing an appropriate agent. However, since a transformant which does not contain the insertion fragment cannot grow when a lethality gene is used, it is not necessary to form colonies on a solid material such as an agar medium, and the selection can be carried out based on the growth by simply culturing in a liquid medium. Accordingly, even in the case of selecting from, for example, 100,000 transformants which are not possible to form colonies on a solid material such as an agar medium, only those which have the insertion fragment can be efficiently concentrated and selected.

An exogenous DNA fragment introduced into a host cell for the purpose of clarifying nucleotide sequence of the introduced DNA fragment or biological function possessed by the DNA fragment, and in the latter case, not only the DNA fragment is simply introduced, but also it is necessary that biological effect by the introduction of the DNA fragment are judged on the chemical factors such as resistance to antibiotics, physical factors such as the ability to grow at a temperature higher than the usual culturing temperature, or other certain factors which can be set. In that case, a DNA fragment having the biological function of interest is selected by using the growing ability of the organism by the intended factor as the index, and in most cases, the discrimination is carried out by setting the above-described factor on a solid medium such as an agar medium and forming colonies thereon, and the DNA fragments possessed by the colonies is analyzed.

However, since a large number of DNA fragments are present which can form the colonies, when preparation of different DNA fragments, for example, from scores to hundreds kinds, is expected, it is necessary to analyze the above-described colonies equal to or larger than the number of expected kinds, generally colonies of at least 10 times to 100 times larger numbers than the number, by a method such as DNA sequencing. On the other hand, in the recent years with advanced analytical techniques in terms of genomic science, it is possible to analyze a large number of DNA fragments in one lot, for example, the above-described DNA fragments possessed by the colonies can be analyzed by using a DNA microarray having several thousand or more kinds of different nucleotide sequences. In that case, according to the conventional methods, the following two types of methods are applied to the method for preparing samples to be analyzed.

In the first method, a sample is prepared from a transformant in the form of a plasmid or the like in the state of containing an insertion DNA fragment, treated with an appropriate labeling such as a fluorescence labeling, and then analyzed by the DNA microarray. In this case, since a large amount of DNA unnecessary for the analyst which is derived from a vector such as a plasmid is present in addition to the insertion fragment necessary for the hybridization of the DNA microarray, the contamination with a large amount of unnecessary labeled DNA fragments causes increase of the background and leads to decrease of the signal/noise ratio. In addition, separation and purification of a large amount of DNA are required in order to ensure sufficient sensitivity.

In the second method, PCR can be used for the purpose of improving the problems of the first method. In this case, a set of PCR primers interposing the insertion fragment are designed based on vector-derived nucleotide sequences in the vicinity of the insertion fragment, and PCR of all insertion fragments is carried out in one lot using DNA extracted from a group of the above-described colonies as the template. In parallel with the PCR reaction, or after the PCR reaction, a DNA fragment as the PCR product is labeled with fluorescence or the like and analyzed by the DNA microarray. According to this method, the vector-derived DNA moiety contaminated in the amplification product can be limited to a markedly small amount, with the necessary part for the above-described primers as the minimum, so that a high signal/noise ratio can be realized. Also, according to this method, since amplification by PCR is possible, the above-described preparation of DNA from a group of colonies is sufficient in a small amount so that a high detection sensitivity can be conveniently realized. However, a vector having no insertion fragment is also amplified as a template by the above-described PCR, but the amplified fragment becomes a short DNA fragment of generally one/several parts or less in length, in comparison with the amplified fragment derived from a vector containing the insertion fragment. Since a shorter DNA fragment is amplified by the PCR amplification with a high efficiency in comparison with a longer DNA fragment, contamination with a large amount of the short DNA fragment which is not an object of the analysis is induced by the presence of a vector which does not have the insertion fragment. In addition, since the substrate necessary for the PCR amplification is consumed for the amplification of the useless short DNA fragment which does not have the insertion fragment, amplification of the insertion fragment necessary for the analysis is considerably obstructed. As a result, both of the signal/noise ratio and detection sensitivity are spoiled.

When the vector of the present invention is used, transformants having no insertion fragment can be removed almost completely. Accordingly, reduction of both of the signal/noise ratio and detection sensitivity as the problem of the second conventional method can be sharply improved. In addition, since the selection marker of the present invention has lethality, it is possible to selectively concentrate the candidates not only on a solid medium such as an agar medium but also in the state of liquid culture. Accordingly, as the selection of transformants, it is possible to select a hundred thousand or more of transformants, which is generally impossible on a solid medium, so that a comprehensive analysis can be realized on a large number of genes, which is impossible so far, such as screening from organisms having a large genomic size such as human, screening of cDNA derived from a gene having low expression frequency and the like.

### EXAMPLE 1

A DNA fragment containing the CRD region (ref) of colicin E3 was amplified by PCR using primers represented by SEQ ID NO:1 and SEQ ID NO:2, and a DNA fragment containing the immunity (ref.) of the same using primers represented by SEQ ID NO:3 and SEQ ID NO:4, from an E. coli colicin E3 plasmid (pSH350) (which has been deposited on Jury 25, 2003, as FERM BP-8436 in International Patent Organism Depositary, National Institute of Advanced Industrial Science and Technology (Central 6, 1-1, 1-Chome, Tsukuba-shi, Tbaraki-ken, Japan). Next, a DNA fragment represented by SEQ ID NO:7 was obtained by carrying out PCR, using a fragment prepared by fusing both of the fragments as the template and using primers represented by SEQ ID NO:5 and SEQ ID NO:6. The structure of this DNA fragment is shown below.

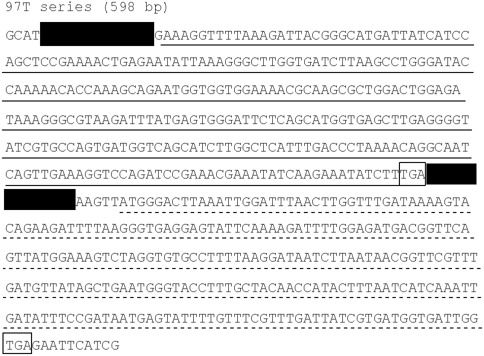

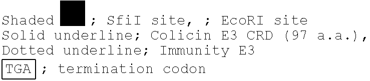

Next, TA cloning of the DNA fragment was carried out by using pGEM T easy vector (manufactured by Promega), and a plasmid pGEM-97col+imm having an insertion fragment of a correct nucleotide sequence was obtained by sequence analysis. In this connection, the colicin E3 immunity gene was used to stably maintain the CRD region of colicin E3 on the plasmid.

Next, fragments amplified by using the primers represented by SEQ ID NO:8 and SEQ ID NO:6 was subjected to PCR by using the just described plasmid as the template and further using primers represented by each of SEQ ID NO:9 to SEQ ID NO:13 and SEQ ID NO:6 to thereby obtain DNA fragments having 1 to 5 amber termination codons (TAG) in just upstream of the CRD region of colicin E3 and the above-described colicin E3 immunity gene in the downstream. Among these, the structure of a DNA fragment (SEQ ID NO:14) into which 3 amber termination codons were inserted is shown below.

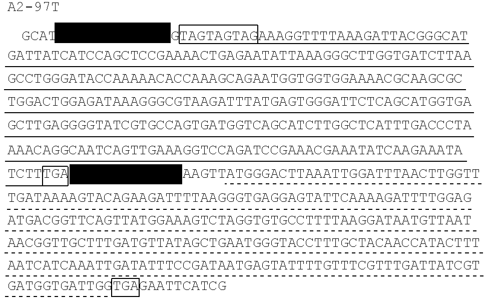

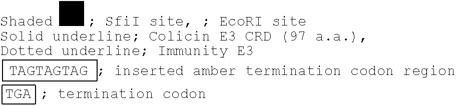

Each of these DNA fragments having 1 to 5 amber termination codons was subjected to TA cloning using pGEM T easy vector (manufactured by Promega), and then to sequence analysis to obtain 5 plasmids having respective insertion fragments of correct nucleotide sequences, namely pCI3A1 (which has been deposited on Jury 24, 2003, as FERM BP-8437 in International Patent Organism Depositary, National Institute of Advanced Industrial Science and Technology (Central 6, 1-1, 1-Chome, Tsukuba-shi, Ibaraki-ken, Japan), pCI3A2 (which has been deposited on Jury 24, 2003, as FERM BP-8438 in International Patent Organism Depositary, National Institute of Advanced Industrial Science and Technology (Central 6, 1-1, 1-Chome, Tsukuba-shi, Ibaraki-ken, Japan), pCI3A3 (which has been deposited on Jury 24, 2003, as FERM BP-8439 in International Patent Organism Depositary, National Institute of Advanced Industrial Science and Technology (Central 6, 1-1, 1-Chome, Tsukuba-shi, Ibaraki-ken, Japan), pCI3A4 (which has been deposited on Jury 24, 2003, as FERM BP-8440 in International Patent Organism Depositary, National Institute of Advanced Industrial Science and Technology (Central 6, 1-1, 1-Chome, Tsukuba-shi, Ibaraki-ken, Japan) and pCI3A5 (which has been deposited on Jury 24, 2003, as FERM BP-8441 in International Patent Organism Depositary, National Institute of Advanced Industrial Science and Technology (Central 6, 1-1, 1-Chome, Tsukuba-shi, Tbaraki-ken, Japan). On the other hand, as the vector, a plasmid pBS2SKP-SfiI into which two SfiI cleavage sites (shown by underlines) having different protruding terminal sequences were inserted was constructed by annealing two synthetic single-stranded oligonucleotides represented by SEQ ID NO:21 and SEQ ID NO:22, and inserting the thus formed double-stranded DNA fragment between Bamfi and EcoRI of pBluescript II SK(+). This plasmid was digested with SfiI, ligated with the above-described colicin E3 CRD gene fragments having 1 to 3 amber termination codons, and then transformed into an E. coli strain XL1-Blue by electroporation. As a result of spreading the thus obtained E. coli cell suspension on an agar medium containing 100 mg/l ampicillin and 0.1% glucose and culturing at 37° C. for a whole day and night, transformants were obtained on the agar medium only in the case in which three amber termination codons were inserted. When plasmid pBS-Sfi-a3col was recovered from the thus obtained transformants and transformed into XL1-Blue, and then the resulting E. coli cell suspension was spread on an agar medium containing 100 mg/l ampicillin+0.1% glucose, and on an agar medium containing 100 mg/l ampicillin+200 μM IPTG (isopropyl-β-D-thiogalactopyranoside) and cultured at 37° C. for a whole day and night, a large number of colonies were formed only when cultured on the medium containing glucose, and formation of colonies was not found on the medium containing IPTG.

### EXAMPLE 2

Two double-stranded DNA fragments GAL4DBD and ENOAPL represented by SEQ ID NOs:23 and 24 were prepared, mixed with a DNA fragment prepared by digesting the plasmid pBS-Sfi-a3col prepared in Example 1 with SfiI to carry out ligation reaction with a DNA ligase, and then transformed into the E. coli strain XL-Blue. When clones of the thus obtained transformants were optionally selected to recover plasmids, and then the inserted DNA fragments were analyzed, 10 to 30% of clones having no insertion fragment were present in the presence of glucose, while clones having no insertion fragment were not detected when grown in the presence of IPTG. Expression of a lethal gene by modification of colicin E3 inserted into a vector is inhibited in the presence of glucose by the controllable promoter positioned at its upstream, but is induced in the presence of IPTG. Thus, it was shown that clones having no insertion fragment can be completely excluded by setting a condition under which the lethal gene can be expressed. In this connection, when the termination codon was not inserted by the present invention, the control at the transcriptional regulation by this Example was impossible, and it could not be maintained stably in E. coli as the host. In this case, a trouble such as the use of an E. coli strain containing the immunity E3 gene becomes necessary in order to amplify the vector DNA.

Based on the above, it was shown that the SfiI-digested DNA fragment represented by SEQ ID NO:14 can function as a lethality marker for cloning an exogenous DNA fragment at a high efficiency, and that a plasmid vector into which this fragment was inserted can be used as for exogenous DNA fragment cloning.

TABLE 1

Insertion DNA fragment

| | GAL4DBD | ENOAPL |
|---|---|---|
| IPTG (+) | 21 (0) | 18 (0) |
| Glucose (+) | 20 (8) | 19 (2) |

The number of clones having or not having insertion fragment

The numerical value in the table indicates the number of clones having insertion fragment among the analyzed transformant clones, and the value in parentheses indicates the number of clones having no insertion fragment.

### INDUSTRIAL APPLICABILITY

According to the present invention, a markedly effective means can be provided for efficiently selecting a clone having an exogenous insertion gene fraction, in carrying out transformation using a lethal gene such as of colicin. Particularly, the transformant selection marker of the present invention can be freely constructed and selected in response to the degree of lethal activity of the lethal gene to be used the strength of suppressor mutation possessed by the host to be used, so that it becomes possible to construct and select a selection marker most efficient for the host to be used, reduction of selectivity based on the resistance acquirement by the host due to too strong lethal activity of a lethal gene can be prevented, and a vector containing the selection marker can be amplified stably in the host. In addition, it is possible to stably amplify it in the same manner, by further adding a gene having resistance to a lethal gene such as immunity to the active moiety of the lethal gene, or by keeping a plasmid having such a resistant gene in advance in a host E. coli. Accordingly, the present invention provides a means markedly useful as a means for cloning exogenous insertion gene.

1: A DNA fragment in which a translation termination codon is inserted into the 5′ upstream side of an active site of a lethal gene.

2: The DNA fragment according to claim 1, which has restriction enzyme cleavage sites in both terminal sides.

3: The DNA fragment according to claim 2, wherein one or at least two translation termination codons are inserted.

4: The DNA fragment according to claim 1, wherein the active site encodes a colicin-derived polypeptide.

5: The DNA fragment according to claim 1, wherein the active site comprises a nucleotide sequence encoding the amino acid sequence represented by SEQ ID NO:18 or 19.

6: A DNA fragment which comprises the nucleotide sequence represented by SEQ ID NO:14.

7: The DNA fragment according to claims 1 or 6, wherein a neutralizing gene for the lethal gene is conjugated to the 3′ downstream side of the active site of the lethal gene.

8: The DNA fragment according to claim 7, wherein the nucleotide sequence of the neutralizing gene is represented by SEQ ID NO:15.

9: A marker for transformant selection, which comprises the DNA fragment according to claim 1 or 6.

10: The marker for transformant selection according to claim 9, wherein the transformant is obtained by transforming Escherichia coli.

11: A recombinant vector into which the DNA fragment according to claim 1 or 6 is inserted.

12: The recombinant vector according to claim 11, which is free of an expression promoter for the lethal gene.


SEQUENCE LISTING (24 sequences)

SEQ ID NO: 1 - DNA, 28 bp, Artificial sequence
  Primer
gctgatgctgcattgagttctgctatgg

SEQ ID NO: 2 - DNA, 57 bp, Artificial sequence
  Primer
gttaaatccaatttaagtcccataacttggccgctatggcctcaaagatatttcttg

SEQ ID NO: 3 - DNA, 57 bp, Artificial sequence
  Primer
caagaaatatctttgaggccatagcggccaagttatgggacttaaattggatttaac

SEQ ID NO: 4 - DNA, 28 bp, Artificial sequence
  Primer
tcatccctgataatatttgatcaccaat

SEQ ID NO: 5 - DNA, 43 bp, Artificial sequence
  Primer
gcatggccgcctcggccgaaaggttttaaagattacgggcatg

SEQ ID NO: 6 - DNA, 34 bp, Artificial sequence
  Primer
cgatgaattctcaccaatcaccatcacgataatc

SEQ ID NO: 7 - DNA, 598 bp, Escherichia coli
gcatggccgcctcggccgaaaggttttaaagattacgggcatgattatcatccagctccgaaaactgagaatattaaagggcttggtgatcttaagcctgggataccaaaaacaccaaagcagaatggtggtggaaaacgcaagcgctggactggagataaagggcgtaagatttatgagtgggattctcagcatggtgagcttgaggggtatcgtgccagtgatggtcagcatcttggctcatttgaccctaaaacaggcaatcagttgaaaggtccagatccgaaacgaaatatcaagaaatatctttgaggccatagcggccaagttatgggacttaaattggatttaacttggtttgataaaagtacagaagattttaagggtgaggagtattcaaaagattttggagatgacggttcagttatggaaagtctaggtgtgccttttaaggataatgttaataacggttgctttgatgttatagctgaatgggtacctttgctacaaccatactttaatcatcaaattgatatttccgataatgagtattttgtttcgtttgattatcgtgatggtgattggtgagaattcatcg

SEQ ID NO: 8 - DNA, 40 bp, Artificial sequence
  Primer
tagtagtagtagtagaaaggttttaaagattacgggcatg

SEQ ID NO: 9 - DNA, 46 bp, Escherichia coli
gcatggccgcctcggccgtagaaaggttttaaagattacgggcatg

SEQ ID NO: 10 - DNA, 49 bp, Artificial sequence
  Primer
gcatggccgcctcggccgtagtagaaaggttttaaagattacgggcatg

SEQ ID NO: 11 - DNA, 52 bp, Artificial sequence
  Primer
gcatggccgcctcggccgtagtagtagaaaggttttaaagattacgggcatg

SEQ ID NO: 12 - DNA, 55 bp, Artificial sequence
  Primer
gcatggccgcctcggccgtagtagtagtagaaaggttttaaagattacgggcatg

SEQ ID NO: 13 - DNA, 58 bp, Artificial sequence
  Primer
gcatggccgcctcggccgtagtagtagtagtagaaaggttttaaagattacgggcatg

SEQ ID NO: 14 - DNA, 607 bp, Escherichia coli
gcatggccgcctcggccgtagtagtagaaaggttttaaagattacgggcatgattatcatccagctccgaaaactgagaatattaaagggcttggtgatcttaagcctgggataccaaaaacaccaaagcagaatggtggtggaaaacgcaagcgctggactggagataaagggcgtaagatttatgagtgggattctcagcatggtgagcttgaggggtatcgtgccagtgatggtcagcatcttggctcatttgaccctaaaacaggcaatcagttgaaaggtccagatccgaaacgaaatatcaagaaatatctttgaggccatagcggccaagttatgggacttaaattggatttaacttggtttgataaaagtacagaagattttaagggtgaggagtattcaaaagattttggagatgacggttcagttatggaaagtctaggtgtgccttttaaggataatgttaataacggttgctttgatgttatagctgaatgggtacctttgctacaaccatactttaatcatcaaattgatatttccgataatgagtattttgtttcgtttgattatcgtgatggtgattggtgagaattcatcg

SEQ ID NO: 15 - DNA, 258 bp, Escherichia coli
atgggacttaaattggatttaacttggtttgataaaagtacagaagattttaagggtgaggagtattcaaaagattttggagatgacggttcagttatggaaagtctaggtgtgccttttaaggataatgttaataacggttgctttgatgttatagctgaatgggtacctttgctacaaccatactttaatcatcaaattgatatttccgataatgagtattttgtttcgtttgattatcgtgatggtgattggtga

SEQ ID NO: 16 - DNA, 3066 bp, Escherichia coli
aactcggttttaatcagacctggcatgagtggaagcgggacgaacagcacaggcaacaacaacgccgccccgggcacttccggggcatgagtatgtgatatccggggctgcaccccggaccccgccaacacatcacgggccacaaaattttttgtggcccgctctgcgttttctaagtgttatccctcctgatttctaaaaaattttccacctgaacttgacagaaaaaacgatgacgagtactttttgatctgtacataaacccagtggttttatgtacagtattaatcgtgtaatcaattgttttaacgcttaaaagagggaatttttatgagcggtggcgatggacgcggccataacacgggcgcgcatagcacaagtggtaacattaatggtggcccgaccgggcttggtgtaggtggtggtgcttctgatggctccggatggagttcggaaaataacccgtggggtggtggttccggtagcggcattcactggggtggtggttccggtcatggtaatggcggggggaatggtaattccggtggtggttcgggaacaggcggtaatctgtcagcagtagctgcgccagtggcatttggttttccggcactttccactccaggagctggcggtctggcggtcagtatttcagcgggagcattatcggcagctattgctgatattatggctgccctgaaaggaccgtttaaatttggtctttggggggtggctttatatggtgtattgccatcacaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgagcgacagaatatttcggttgtttcaggtgttccgatgagtgttccggtggttgatgcaaaacctaccgaacgtccgggtgtttttacggcatcaattccaggtgcacctgttctgaatatttcagttaataacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagccctgaccaggtaaaacaacgtcaagatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagcccagcgggcgcagacggatgtaaataataagcaggctgcatttgatgctgctgcaaaagagaagtcagatgctgatgctgcattgagttctgctatggaaagcaggaagaagaaagaagataagaaaaggagtgctgaaaataatttaaacgatgaaaagaataagcccagaaaaggttttaaagattacgggcatgattatcatccagctccgaaaactgagaatattaaagggcttggtgatcttaagcctgggataccaaaaacaccaaagcagaatggtggtggaaaacgcaagcgctggactggagataaagggcgtaagatttatgagtgggattctcagcatggtgagcttgaggggtatcgtgccagtgatggtcagcatcttggctcatttgaccctaaaacaggcaatcagttgaaaggtccagatccgaaacgaaatatcaagaaatatctttgagaggaagttatgggacttaaattggatttaacttggtttgataaaagtacagaagattttaagggtgaggagtattcaaaagattttggagatgacggttcagttatggaaagtctaggtgtgccttttaaggataatgttaataacggttgctttgatgttatagctgaatgggtacctttgctacaaccatactttaatcatcaaattgatatttccgataatgagtattttgtttcgtttgattatcgtgatggtgattggtgatcaaatattatcagggatgagttgatatacgggcttctagtgttcatggatgaacgctggagcctccaaatgtagaaatgttatattttttattgagttcttggttataattgctccgcaatgatttaaataagcattatttaaaacattctcaggagaggtgaaggtggagctaaaaaaaagtattggtgattacactgaaaccgaattcaaaaaatttattgaagacatcatcaattgtgaaggtgatgaaaaaaaacaggatgataacctcgagtattttataaatgttactgagcatcctagtggttctgatctgatttattacccagaaggtaataatgatggtagccctgaaggtgttattaaagagattaaagaatggcgagccgctaacggtaagtcaggatttaaacagggctgaaatatgaatgccggttgtttatggatgaatggctggcattctttcacaacaaggagtcgttatgaaaaaaataacagggattattttattgcttcttgcagtcattattctgtctgcatgtcaggcaaactatatccgggatgttcagggcgggaccgtatctccgtcatcaacagctgaagtgaccggattagcaacgcagtaacccgaaatcctctttgacaaaaacaaagcgtgtcaggctgattctgatgcgctttttttttgaaatgtcacaaaaattccatgtgggagatgggatctaaaatcctcgtgcagaactttccatccagggggagaaaacttgtcgttttgagccgttcggtgttcagaacgcacgaaaccgatcg

SEQ ID NO: 17 - protein, 551 aa, Escherichia coli
MSGGDGRGHNTGAHSTSGNINGGPTGLGVGGGASDGSGWSSENNPWGGGSGSGIHWGGGSGHGNGGGNGNSGGGSGTGGNLSAVAAPVAFGFPALSTPGAGGLAVSISAGALSAAIADIMAALKGPFKFGLWGVALYGVLPSQIAKDDPNMMSKIVTSLPADDITESPVSSLPLDKATVNVNVRVVDDVKDERQNISVVSGVPMSVPVVDAKPTERPGVFTASIPGAPVLNISVNNSTPAVQTLSPGVTNNTDKDVRPAGFTQGGNTRDAVIRFPKDSGHNAVYVSVSDVLSPDQVKQRQDEENRRQQEWDATHPVEAAERNYERARAELNQANEDVARNQERQAKAVQVYNSRKSELDAANKTLADAIAEIKQFNRFAHDPMAGGHRMWQMAGLKAQRAQTDVNNKQAAFDAAAKEKSDADAALSSAMESRKKKEDKKRSAENNLNDEKNKPRKGFKDYGHDYHPAPKTENIKGLGDLKPGIPKTPKQNGGGKRKRWTGDKGRKIYEWDSQHGELEGYRASDGQHLGSFDPKTGNQLKGPDPKRNIKKYL

SEQ ID NO: 18 - protein, 110 aa, Escherichia coli
AENNLNDEKNKPRKGFKDYGHDYHPAPKTENIKGLGDLKPGIPKTPKQNGGGKRKRWTGDKGRKIYEWDSQHGELEGYRASDGQHLGSFDPKTGNQLKGPDPKRNIKKYL

SEQ ID NO: 19 - protein, 97 aa, Escherichia coli
KGFKDYGHDYHPAPKTENIKGLGDLKPGIPKTPKQNGGGKRKRWTGDKGRKIYEWDSQHGELEGYRASDGQHLGSFDPKTGNQLKGPDPKRNIKKYL

SEQ ID NO: 20 - DNA, 330 bp, Escherichia coli
ggccgcctcggccgtagtagtagaaaggttttaaagattacgggcatgattatcatccagctccgaaaactgagaatattaaagggcttggtgatcttaagcctgggataccaaaaacaccaaagcagaatggtggtggaaaacgcaagcgctggactggagataaagggcgtaagatttatgagtgggattctcagcatggtgagcttgaggggtatcgtgccagtgatggtcagcatcttggctcatttgaccctaaaacaggcaatcagttgaaaggtccagatccgaaacgaaatatcaagaaatatctttgaggccatagcggcc

SEQ ID NO: 21 - DNA, 60 bp, Artificial sequence
  Synthetic single-stranded oligonucleotide
gatccccgggtaccgaggccgcctcggccgagctcgaattcggccggccatagcggccgc

SEQ ID NO: 22 - DNA, 60 bp, Artificial sequence
  Synthetic single-stranded oligonucleotide
aattgcggccgctatggccggccgaattcgagctcggccgaggcggcctcggtacccggg

SEQ ID NO: 23 - DNA, 650 bp, Saccharomyces cerevisiae
ggccgcctcggccaggatctggtggcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgattgactcggcagctcatcatgataactccacaattccgttggattttatgcccagggatgctcttcatggatttgattggtctgaagaggatgacatgtcggatggcttgcccttcctgaaaacggaccccaacaataatgggttctttggcgacggttctctcttatgtattcttcgctgactgactgaggccatagcggcc

SEQ ID NO: 24 - DNA, 535 bp, Aspergillus oryzae
ggccgcctcggccattactagtctactagtaactctgtcttatcgtcatctcccataggtgagtttggttgttttgtttccactgagatcatgacctcctcctaccccaccatcccactatttttgttacggtagccatgacccctccatggcaaagagagaggaggacgaggacgatcaggaaactgtgtctcgccgtcataccacaatcgtgttatcctgattgacatcttcttaaatatcgttgtaactgttcctgactctcggtcaactgaaattggatctccccaccactgcctctaccttgtactccgtgactgaaccatccgatcattctttttgggtcgtcggtgaacacaacccccgctgctagtctccttccaacaccgatccagaattgttttgattttccattcccttcgtttatatctgtcgtctctcctccctttccgtctcttttcttccgtcctccaagttagtcgactgaccaattccgcagctcgtcaaaatgcctatcaccaaggccatagcggcc